(19) United States
(12) Patent Application Publication
(10) Pub. No.: US 2026/0215900 A1
(43) Pub. Date: Jul. 30, 2026

(54) SURGICAL CASSETTE

(71) Applicant: INSTITUT STRAUMANN AG, Basel (CH)

(72) Inventor: Silvio BLUMENTHAL, Basel (CH)

(73) Assignee: INSTITUT STRAUMANN AG, Basel (CH)

(21) Appl. No.: 19/149,009

(22) PCT Filed: Jan. 19, 2024
(86) PCT No.: PCT/EP2024/051208

§ 371 Date: Jul. 17, 2025

(30) Foreign Application Priority Data
Jan. 20, 2023 (EP) 23152751.6

(51) Int. Cl. A61C 19/02; A61C 19/00

(52) U.S. Cl. CPC A61C 19/02; A61C 19/002

(57) ABSTRACT

A metal cassette for housing at least one surgical instrument, the cassette including a base component and a lid fastenable to the base component to form an enclosed space in which the at least one surgical instrument can be housed. At least one of base component and lid includes a metal sheet, enclosed space being partly delimited by a boundary forming surface of metal sheet including at least one integral tongue forming at least one holding means for retaining the at least one surgical instrument within the cassette. The tongue includes a section of the metal sheet which is partially detached from metal sheet and is integrally connected to boundary forming surface at least one attachment location, wherein, in a resting state, tongue extends from at least one attachment location into the enclosed space.

The invention relates to a cassette for housing—in particular for storage, surgical use, cleaning and sterilization of—at least one surgical instrument, in particular a dental surgical tool.

Instruments for dental surgery must be carefully cleaned, sterilised, and stored after every use. Increasingly, metal cassettes are used to house these instruments, as such cassettes enable the instruments to be easily cleaned and sterilised within the cassette, for instance by Automated Cleaning and Disinfection (ACD) machines. For hygiene reasons, these cassettes should be free from non-metal components, such as plastic grommets or seals, and contain a minimal amount of grooves, indents etc, as such features are harder to clean and dry and also provide a possibility for water, dirt and bacteria traps.

WO 2016/142331 discloses a metal cassette having one or more sheets which contain integral spring elements for holding instruments in the cassette. Such integral spring elements assist in achieving the above objectives.

There remains a need in the field of surgical cassettes to create a cassette that has the minimum number of parts. Every component which must be attached to the cassette results in some form of joint, which can form a dirt and bacteria trap.

Consequently, it is the object of at least a preferred embodiment of the present invention to provide a surgical cassette which allows safe holding and transport of at least one surgical instrument, and which allows easy and effective cleaning and sterilization of the cassette and its contents.

This object is achieved by the metal cassette as claimed in claim 1. Preferred embodiments are subject of the dependent claims.

The metal cassette of the present invention is intended for housing at least one surgical instrument, and comprises a base component and a lid fastenable to said base component to form an enclosed space in which the at least one surgical instrument can be housed. At least one of said base component and lid comprises a metal sheet, the enclosed space being partly delimited by a boundary forming surface of the metal sheet.

The metal sheet comprises at least one integral tongue forming at least one holding means for retaining the at least one surgical instrument within the cassette, said tongue consisting of a section of the metal sheet which is partially but not fully detached from the sheet and which is integrally connected to the boundary forming surface at at least one attachment location, wherein, in a resting state, the tongue extends from the at least one attachment location into said enclosed space.

Within the context of this application, the “enclosed space” refers to a space between the upper horizontal surface of the base component and the lower horizontal surface of the lid, this space being laterally bordered by at least two opposing walls or other boundaries that are part of the cassette. Preferably, the enclosed space is laterally surrounded by a boundary structure, e.g. a wall, on all sides. However, it does not require a hermetic sealing, as there are generally gaps or holes in these surface and boundary structures to allow cleaning and sterilisation fluids to pass into and through the cassette.

All references to horizontal and vertical orientations, as well as directional terms such as “upper”, “lower”, “side” etc, refer to the orientation and location of the components when the cassette is positioned on a horizontal flat surface in its correct orientation, i.e. with base component positioned closer to the flat surface than the lid.

Within the context of the present invention a “sheet” refers to a component whose thickness is considerably less than, e.g. less than half, its width and length. Typically the thickness of the metal sheet of the present invention is between 0.5 mm and 3 mm. Additionally or alternatively, the thickness of a sheet typically remains constant along its full length and width.

In accordance with the present invention, the at least one tongue retains the at least one instrument within the cassette. It thus forms a holding means for the instrument. The tongue is an integral part of the metal sheet, meaning that it is part of the monolithic structure of the sheet. The tongue does thus not need to be joined to the sheet via, e.g. rivets, welding etc. Creating an integral tongue as proposed by the present invention reduces the number of individual components necessary and thus avoids any joint, seal or other mechanism required to connect the tongue and the sheet together.

Within the context of the present invention, the term “tongue” refers to a section of the metal sheet which is partially, but not fully, detached from the metal sheet, and which is integrally connected to the boundary forming surface at at least one attachment location. As the tongue is an integral part of the sheet the terms “connected” and “attachment” do not refer to the act or process of joining the tongue to the sheet. Instead, these terms are intended only to describe the position of the tongue in relation to the boundary forming surface of the metal sheet.

The partially detached nature of the tongue enables this to extend from the attachment location(s) into the enclosed space delimited (at least partly) by the boundary forming surface. In this way, one or more holding means are provided within the enclosed space of the cassette without the need for any joint, seal or other connection mechanism. The cassette therefore provides increased opportunities for holding surgical instruments while maintaining the simplicity and cleanability of the cassette.

According to the present invention the at least one tongue extends into the enclosed space when it is in its resting state. By “resting state” it is meant the position of the tongue when no external forces are acting on this, for example as might be felt when retaining an instrument.

According to the present invention, the at least one tongue extends from the at least one attachment location with the boundary forming surface into the enclosed space formed between the lid and base component. In other words, the at least one tongue is not in alignment with the boundary forming surface of the metal sheet.

The at least one tongue can extend from the at least one attachment location at any desired angle, but preferably extends at an angle of between 30°-150° relative to the plane of the metal sheet at the at least one attachment location of the tongue. This creates adequate free space around the tongue for the positioning of an instrument. Notably, within the context of the present invention, the term “attachment location” refers to the location(s) of attachment of the tongue to the boundary forming surface. This can take the form of a single attachment location or attachment at several discrete attachment locations.

Most preferably, the at least one tongue extends at an angle of approximately 90° relative to the plane of the metal sheet at the at least one attachment location such that, at its attachment location(s), the tongue extends away from said boundary forming surface in an essentially perpendicular manner.

Throughout this specification, the term “approximately” is used to cover not just the stated angle or orientation, but also minor deviations from this that may arise during manufacturing, but which fall within accepted tolerance limits, e.g. ±5°. Thus, an angle of “approximately 90°” refers to angles between 85-95°.

According to the present invention the at least one tongue is partially but not fully detached from the metal sheet and extends from the boundary forming surface into the enclosed space of the cassette.

According to standard manufacturing procedures, this can most easily be achieved by creating a partially detached cut out in the metal sheet via, e.g., laser cutting or water jet cutting. This cut out is then bent out of alignment with the metal sheet to form the tongue. This production method is simple and cost effective, and in addition the hole left behind in the boundary forming surface after formation of the tongue provides a passage for sterilising steam in the vicinity of the tool held by the tongue. In addition, creating a holding means by removal of material, rather than addition of a further component, is an elegant method of providing retention without complexity or introducing dirt, water and bacteria traps.

Although the above-mentioned method of production is currently preferred, a sheet having an integral tongue can also be produced using, e.g. injection moulding or 3D printing methods. Thus, it is possible for the at least one tongue to have a bent shape without this being achieved by bending. The term “bent” as used in the remainder of this specification therefore refers to the tongue's orientation relative to the surrounding metal sheet and not to its method of production. Furthermore, when injection moulding or 3D printing is used there is no need to produce a cut out in the sheet. However, for the fluid circulation benefit mentioned above, such a “cut out” geometry may still be present when these manufacturing methods are used. Therefore, use of the term “cut out” within the specification refers to the physical shape of the sheet and not to the production method used.

In a preferred embodiment therefore the at least one tongue is formed by a partially detached cut out in the boundary forming surface of the metal sheet, said cut out being bent out of alignment with the boundary forming surface at the at least one attachment location.

According to the present invention the metal sheet has a boundary forming surface that partly delimits the enclosed space of the cassette. The metal sheet may form the upper or lower boundary of the enclosed space, and/or one or more side boundary of the enclosed space. The one or more tongue can be integrally connected to any part of the boundary forming surface, i.e. on an upper, lower or side boundary of the enclosed space.

In one embodiment it is possible for the metal sheet to comprise a boundary forming surface that comprises a first planar section and a second planar section, with the second planar section positioned at right angles to the first planar section. In this way the metal sheet forms two mutually perpendicular surfaces delimiting the enclosed space. In such embodiments the at least one tongue may be formed by a single cut out created in both the first and the second planar sections, and bent out of both planes so that a non-planar “inverse L-shaped” tongue extends into the enclosed space. Preferably however, the at least one tongue is formed by a cut out in a section of the boundary forming surface which extends in a single plane. Thus, even if the boundary forming surface of the metal sheet comprises two or more sections extending in different planes, the at least one tongue is formed by a cut out in a planar surface. This facilitates manufacturing of the tongue when traditional manufacturing methods are used.

In a preferred embodiment the cut out is fully surrounded by an area of boundary forming surface which extends in a single plane. This reduces the possibility of dust and bacteria traps and increases cleanability in the vicinity of the tongue.

The at least one tongue can be of any shape desired, but preferably comprises a generally polygonal shape wherein one of the sides of the polygon is integrally connected to the boundary forming surface at at least one attachment location. Most preferably the tongue has a generally quadrilateral shape.

It is possible that the tongue is bent at different locations along its length. For example, the distal end of the tongue may be bent at an angle of 90° relative to the proximal end of the tongue in order to form an “inverted L” shape. This could be achieved via the non-planar cut out described above, or through bending an initially planar tongue, or via additive manufacturing. Alternatively, the distal end of the tongue may comprise a bisecting vertical split, enabling the two half areas at the distal end of the tongue to extend at an angle to each other, thus forming a “Y” shaped tongue.

However, a single plane is preferred both for simplicity of manufacturing and also for cleaning purposes. Therefore the at least one tongue preferably extends in a single plane.

As discussed above, the one or more tongue forms at least one holding means for retaining the at least one surgical instrument within the metal cassette. In certain embodiments the tongue may achieve this by holding the instrument against an opposing surface within the cassette. For example, one or more tongue(s) provided on the lid may retain an instrument against the base component, or one or more tongue(s) provided on the base component may retain an instrument against the lid. Alternatively, two tongues on the base component may hold an instrument positioned between the tongues, each tongue acting to press the instrument against a surface of the opposing tongue. In a further alternative, the “inverted L-shaped” tongue discussed above can retain an instrument against the boundary forming surface of the metal sheet from which the tongue extends.

These forms of holding means are referred to herein as “cooperative holding means”, as the tongue does not operate independently to retain an instrument but must cooperate with another surface of the cassette (e.g. the boundary forming surface or the surface of another tongue) to provide a retention function. When the tongue forms a cooperative holding means it is preferable that the at least one tongue forms at least one holding means comprising a holding surface for retaining the instrument, said holding surface opposing a cooperating surface of the cassette not located on said tongue such that in use the instrument is retained between the holding surface and the cooperating surface. In such embodiments it is preferable that the holding surface of the tongue is planar and further preferable that the cooperating surface comprises a cavity, depression or aperture for housing the instrument. This ensures that the instrument is correctly placed in the cassette and also assists in preventing movement of the instrument. Preferably the cavity, depression or aperture is sized to only partially house the instrument. This minimises the surface area of the instrument which contacts or overlaps the cavity, depression or aperture and thus facilitates the cleaning and sterilising of the instrument within the cassette.

In one particularly preferred example of such a holding means, at least one tongue is formed in the lid of the cassette, with a planar holding surface facing the upper horizontal surface of the base component. The upper horizontal surface of the base component in the area opposing the planar holding surface comprises an aperture sized to house a part of a surgical instrument. The surgical instrument is in use placed in the aperture and, when the lid is fastened to the base component, the tongue holds the instrument within this aperture, thus preventing its inadvertent removal.

In contrast it is alternatively possible for the cooperating surface to comprise a planar surface while the holding surface of the tongue comprises a cavity, depression or aperture. For example, the distal end of the tongue may comprise an opening or passage into which a part of the instrument, e.g. a section of the instrument shaft, may be placed. The opposing planar cooperating surface can in use close off the opening or passage of the tongue in order to prevent removal of the instrument from this.

The tongue may provide a passive or active hold on the instrument. By “passive” it is meant that the tongue does not exert a force on the instrument during retention. In contrast, an “active” hold is formed when the tongue exerts a biasing force on the instrument during use. As the at least one tongue is formed of metal, it has an inherent elasticity when deflected from its resting state. This can be used to press the instrument against the opposing surface, e.g. the base component, lid, or other tongue. In the preferred embodiment above, if the gap (i.e. space in-between) provided between the holding surface of the tongue and the opposing cooperating surface is less than the width or diameter of the instrument to be held, then the tongue will be deflected from its resting state by the instrument and a biasing force will push the instrument against the opposing cooperating surface.

However, in preferred embodiments the at least one tongue is arranged to provide a passive hold on the instrument. Thus, in such embodiments the gap provided between the holding surface and the opposing cooperating surface is equal to or slightly greater than the width or diameter of the instrument. In this way no biasing force is exerted on the instrument. This loose hold is beneficial for allowing a good flow of sterilising steam around the entire surface of the instrument.

In the examples of “cooperative holding means” described above, the holding means formed by the tongue cooperates with another surface in the cassette in order to retain the instrument. In other embodiments however, the at least one tongue forms at least one self-sufficient holding means. In such embodiments the tongue is capable of providing retention to a surgical instrument self-sufficiently, without cooperation with any other component or surface of the cassette. Preferably, in such embodiments, the at least one tongue forms at least one holding means comprising at least one integrally formed spring element, moveable relative to the remainder of the tongue, for resiliently retaining an instrument between the at least one spring element and another area of the tongue. By “integral”, as above, it is meant that the spring element is not a separate component that is inserted into or coupled to the tongue, but is part of the monolithic structure of the tongue and hence also the metal sheet. As such, the integral nature of the spring element avoids the use of joints or other (non-integral) connections between the spring element and the tongue that would provide some kind of gap in which water and/or bacteria could accumulate.

The provision of at least one spring element in the tongue enables the tongue to hold an instrument without cooperation with a separate element of the cassette. Instead, the instrument can be held between the spring element and a separate area of the tongue. For example, in the “Y shaped” tongue described above, both arms may form spring elements which each providing an opposing biasing force towards the central axis of the tongue. In this way, the shaft of an instrument positioned between the arms will be held by the opposing forces exerted by the arms; the instrument will thus be retained between the two spring elements.

In the same manner as discussed above in relation to cooperative holding means, the tongue may form a passive or active hold on the instrument. In embodiments in which the tongue comprises at least one spring element, by “passive” it is meant that the at least one spring element does not exert a force on the instrument during retention. In contrast, an “active” hold is formed when the at least one spring element exerts a biasing force on the instrument during use.

When the tongue forms at least one self-sufficient holding means, a passive and thus “loose” hold is again preferred in order to aid cleaning and sterilisation.

In preferred embodiments the at least one spring element consists of a section of the tongue which is partially but not fully detached from the tongue and which is integrally connected to the tongue at a proximal attachment region. While, in a similar manner to the tongue itself in relation to the boundary forming surface, the spring element can be bent out of the plane of the tongue (as in the “Y shaped” example above), preferably the at least one spring element extends in the plane of the tongue, such that the spring element is coplanar to the tongue when it is in the resting state. In this context, the term “in the plane of the tongue” means “the plane of the part of the tongue in which the spring element is formed”.

The at least one spring element can be deflected within and/or out of the plane of the tongue and acts to resiliently retain an instrument within the cassette.

The resting state in this context refers to the position of the spring element when no external forces are acting on this. Contrary thereto, the active state refers to the state of the spring element when deflected from its resting state. While being preferably co-planar to the tongue in its resting state, in the active state, the at least one spring element may be bent out of the plane of the tongue, such that a surgical instrument can be placed under, on top or beside the spring element.

In a particularly preferred embodiment, the at least one holding means further comprises a passage through the thickness of the tongue, the at least one integral coplanar spring element described above being adjacent to the passage. In this way the at least one spring element forms a part of the boundary of the passage. In some embodiments the passage comprises a closed cross-section and can be designed in a similar manner to the holding means disclosed in WO2016/142331. Preferably however the passage includes a distal insertion section that is open towards a distal free end of the tongue for allowing insertion of a surgical instrument into the passage and a proximal holding section intended for releasably housing the surgical instrument. The distal insertion section thus forms an opening through which a surgical instrument can be inserted—preferably laterally. “Proximal” thereby refers to a location closer to the attachment location of the tongue and “distal” consequently to a location further remote from the attachment location.

In this preferred embodiment it is preferable that the tongue extends in a single plane from a horizontal boundary forming surface of the metal sheet. Preferably, the tongue is oriented essentially perpendicular to the horizontal surface. When inserted laterally into the passage therefore, the longitudinal axis of the instrument is thus oriented essentially perpendicular to the plane of the tongue and parallel to the horizontal boundary forming surface. However, depending on the shape of the instrument to be held other positions and orientations of tongue are possible.

If the tongue comprises a passage of the type described above, the at least one spring element can be arranged to actively hold the instrument within the passage by providing a biasing force to push this against an opposing passage wall. In such cases the width of a part of the proximal holding section will be restricted by the at least one adjacent spring element when said spring element is in a resting state, the restricted width of the holding section being less than the width of the insertion section. However, as mentioned above, it is preferred that the tongue is arranged to provide a passive hold on the instrument. Therefore, it is preferred that the minimum width of the insertion section is created by the at least one adjacent spring element when said spring element is in a resting state, the minimum width of the insertion section being less than the maximum width of the holding section. In this embodiment the at least one spring element is elastically deflectable from the resting state into an active state to increase the width of the insertion section. For instance, the at least one spring element may comprise a detent or nub protruding into the insertion section of the passage, and the instrument must be pushed past said detent or nub in order to reach the holding section. In this way, the surgical instrument can be pressed into the insertion section of the passage, causing the at least one spring element to deflect and the nub to move outwards in order to increase the width of the insertion section. When the surgical instrument has passed the detent or nub, the at least one spring element reverts to its resting state, and the user is provided with a physical sensation, e.g. a jolt, snap or reduction in resistance, that informs them that the surgical instrument has now reached the holding section. This design of the tongue prevents the instrument from exiting the passage while avoiding the need for a biasing force to be applied to the instrument to retain it within the holding section.

Preferably, the maximum width of the holding section is larger than the narrowest width of the insertion section when the at least one spring element is in the active state. The width of the insertion section when the at least one spring element is in the active state is determined by the instrument. The at least one spring element will deflect just enough to allow passage of the instrument. After passing the insertion section, the instrument will be provided with additional space in the holding section, such that the instrument has some freedom to move in the holding section but cannot exit therefrom as long as the at least one spring element is in the resting state. This design of the passage prevents the instrument from exiting the passage, while allowing some movement of the instrument within the holding section for facilitated cleaning and sterilisation. Preferably the insertion section has a width which decreases in the proximal direction. This helps to centre and guide the instrument during insertion.

In certain embodiments the at least one spring element can extend to the distal most end of the insertion section. However, preferably the insertion section comprises a section distal of and partially covering the at least one spring element. This protects the spring element(s) from damage.

While the passage can have multiple adjacent spring elements, for example two opposing spring elements, in a preferred embodiment a single integral coplanar spring element is located adjacent to the passage. This provides sufficient retention for the instrument while also simplifying manufacture of the tongue.

According to the present invention, the at least one integral tongue forms at least one holding means for retaining at least one surgical instrument within the cassette. In a preferred embodiment the metal sheet comprises a plurality of integral tongues, each forming at least one holding means for retaining at least one surgical instrument. The plurality of tongues can have any of the preferred features listed above. In addition, a single cassette may comprise tongues corresponding to different preferred embodiments. For example, a cassette according to the present invention may comprise one or more tongue forming at least one cooperative holding means as well as one or more tongue forming at least one self-sufficient holding means. In addition, a single tongue may form at least one cooperative holding means and at least one self-sufficient holding means.

Any or each tongue of the present invention may form a plurality of holding means. The plurality of holding mean can have any of the preferred features listed above. For example, a tongue may comprise a plurality of planar holding surfaces for retaining at least one instrument, said holding surfaces opposing cooperating surfaces of the cassette not located on the tongue. The planar holding surfaces may be continuous with one another or may be separated by segments of the tongue which are non-coplanar with the holding surfaces (e.g. segments which are shaped to form a gully or ridge between adjacent holding surfaces). In addition or alternatively the plurality of planar holding surfaces on a single tongue may be non-co-planar to each other, in order to accommodate differently sized tools.

Alternatively or additionally, a tongue may form a plurality of holding means each comprising a passage through the thickness of the tongue and at least one integral coplanar spring element adjacent to the passage, each holding means intended to hold a separate instrument or separate parts of the same instrument. In such embodiments the passages and their adjacent spring elements may be shaped differently to one another in order to accommodate different instrument shapes or they may have identical shapes to one another, depending on the instruments to be held.

It is further preferred that, in certain embodiments, one and the same instrument can be retained within the cassette by two or more horizontally or vertically spaced tongues forming self-sufficient holding means. Thus, the term “self-sufficient holding means” does not require that a single tongue provides the sole retention to an instrument, only that the retention that is provided by a particular tongue is achieved independently of any components separate from the tongue.

For example, the cassette may comprise two or more tongues that extend in a direction essentially perpendicular to a horizontal boundary forming surface of the metal sheet and are spaced apart from one another. Preferably, the two or more tongues are arranged essentially parallel or at a predefined angle to one other. Each tongue comprises at least one holding means comprising a passage through the thickness of the tongue open to the distal end of the tongue and within which a surgical instrument can be received, and at least one integral coplanar spring element adjacent to the passage. Preferably one or more passage(s) of the first tongue in this embodiment is axially aligned with a passage of at least one further tongue, such that one surgical instrument can be held in a plurality of passages located in multiple tongues. In this way an instrument can be retained at multiple points along its length and is thus prevented from tilting along its longitudinal axis, which could result in interfering with other instruments housed within the cassette.

According to the present invention, at least one of the base component and lid comprises a metal sheet. In a preferred embodiment both the base component and lid comprise a metal sheet in accordance with the present invention. In this way, integral tongues can be provided on both the base component and lid.

Regardless of whether the lid or base component, or both, comprises a metal sheet in accordance with the present invention, said metal sheet preferably comprises a horizontal boundary forming surface, said horizontal boundary forming surface delimiting the full horizontal extent of the enclosed space. In this way, the upper surface of the base component and/or the lower surface of the lid is formed by a single component, thus simplifying construction and reducing the potential for dust and bacteria traps. Although the horizontal boundary forming surface may comprise ridges, grooves etc., it is preferable that the metal sheet forms a planar horizontal boundary forming surface. This improves the ease of cleaning the cassette.

In addition to comprising a horizontal boundary forming surface, the metal sheet preferably further comprises at least two opposing side surfaces, the side surfaces being formed by end portions of the metal sheet extending at approximately 90° to the, preferably planar, horizontal boundary forming surface. In this way, the metal sheet can delimit multiple sides of the enclosed space.

In embodiments in which the lid comprises a metal sheet according to the present invention it is preferred that this metal sheet has a planar horizontal boundary forming surface and at least two, more preferably four, side boundary forming surfaces, each side surface being formed by an end portion of the metal sheet bent at 90° to the plane of the horizontal surface. In this way the lid defines an inner space between the side surfaces and the horizontal surface. In such embodiments, the at least one tongue of the present invention can extend from the horizontal surface and/or from one or more of the side surfaces of the lid. In each case, the tongue extends inwards into the inner space defined by the lid.

It is particularly preferred for the one or more tongue to extend from at least one attachment location on a side surface of the lid. In addition, it is preferred that the one or more tongue is a planar tongue that extends at an angle of approximately 90° relative to the plane of the side surface to which it is integrally connected, and forms a holding means comprising a planar holding surface for retaining an instrument, said planar holding surface opposing a cooperating surface of the base component. The provision of such cooperative holding means on the lid enables the lid to retain instruments positioned on the upper surface of the base component when the cassette is closed, but, when the lid is removed, enables a user to easily access and remove the instruments. Notably, two or more side surfaces may each comprise at least one tongue and/or there may also be at least one tongue extending from a planar horizontal surface of the metal sheet.

In embodiments in which the base component comprises a metal sheet in accordance with the present invention, also in this case it is preferred that the metal sheet comprises a horizontal, preferably planar, boundary forming surface and two or more side surfaces formed by end portions of the metal sheet bent at approximately 90° from the plane of the horizontal surface. The side surfaces can in this case extend upwards, above the horizontal surface of the sheet. In this way the side surfaces of the base component form a part of the boundary forming surface and help to define the enclosed space between the base component and lid. One or more tongue according to the present invention can be formed on such side surfaces of the base component.

In other embodiments, however, the side surfaces can extend downwards, below the horizontal surface of the sheet. Such side surfaces may help to define a second enclosed space within which instruments, or parts of instruments, can be stored. Additionally or alternatively, such downwardly extending side surfaces can act as support legs for the base component. When the base component comprises such downwardly extending side surfaces, the base component may further comprise a second metal sheet that is positioned to close off the second enclosed space and thus forms a lower surface of the base component. This surface can be a solid or “closed” surface or it may preferably comprise a plurality of through holes to assist with movement of cleaning fluids and sterilising steam through the cassette.

When the base component comprises a metal sheet in accordance with the present invention, it is preferable that at least one tongue extends from the horizontal boundary forming surface of the sheet.

In a particularly preferred embodiment, the base component and lid each comprise a metal sheet in accordance with the present invention. The lid consists of a metal sheet forming a planar horizontal surface and four side surfaces, said side surfaces being formed by end portions of the metal sheet extending at approximately 90° to the horizontal surface, the horizontal and side surfaces forming a boundary forming surface. The base component comprises a metal sheet forming a planar horizontal boundary forming surface and at least two opposing side surfaces, said side surfaces being formed by end portions of the metal sheet extending at approximately 90° to the horizontal surface, said side surfaces extending downwards, i.e. away from the enclosed area between the base component and lid. At least one integral tongue is integrally connected to the horizontal boundary forming surface of the base component and extends from the at least one attachment location into the enclosed space in an essentially perpendicular manner relative to the horizontal boundary forming surface. At least one further integral tongue is integrally connected to a side boundary forming surface of the lid and extends from the at least one attachment location into the enclosed space in an essentially perpendicular manner relative to the side boundary forming surface. Preferably the at least one tongue connected to the horizontal boundary forming surface of the base component forms a self-sufficient holding means having the preferred features discussed above. In particular, the one or more tongue preferably extends in a single plane and comprises one or more holding means comprising a passage through the thickness of the tongue and an integral co-planar spring element adjacent to the passage. Preferably the at least one further tongue connected to the side boundary forming surface of the lid forms a cooperative holding means having the preferred features discussed above. In particular, it is preferred that the at least one further tongue comprises a planar holding surface opposing a cooperating surface of the horizontal boundary forming surface of the base component, the cooperating surface comprising an aperture sized to partially house a surgical instrument.

The metal sheet(s) of the present invention preferably further comprise a plurality of through holes for passage of cleaning and sterilizing fluids. The through holes can be provided in any shape and pattern.

In addition to the lid and base component the surgical cassette may comprise additional components, for example a secondary base component that is positioned essentially parallel to the first base component and can be fastened to the underside of the first base component in order to increase the number of tools that can be retained within the cassette.

For example, the cassette may comprise a second base component formed of a horizontal metal sheet with two or more side surfaces bent out of the plane of the sheet in an upwards direction, the first base component of the surgical cassette being supported on the side surfaces of the second base component.

In such instances the first base component also functions as a lid in relation to the second base component, creating an enclosed space in which tongues in accordance with the present invention can be located. Therefore, in the context of the present invention, a “lid” does not need to be the uppermost component of the cassette but simply a component positioned above the base component which can be releasably fastened thereto to create an enclosed space for the housing of at least one instrument.

As discussed above, it is preferred that the lid of the inventive cassette comprises a metal sheet according to the present invention, whereby the metal sheet comprises a planar horizontal surface and two or more side surfaces, the side surfaces being formed by end portions of the metal sheet bent at 90° to the plane of the horizontal surface, the horizontal and side surfaces forming a boundary forming surface of the enclosed space. It is further preferable that the lid further comprises one or more integral clasp, the clasp being formed by at least part of a side surface of the lid, wherein the clasp is pivotable relative to the horizontal surface, the clasp comprising a catch, said catch being formed by a portion of the metal sheet which is bent out of the plane of the clasp so as to form an upwardly facing surface. In such embodiments the base component preferably comprises one or more side walls, one or more sidewall (s) comprising a downwardly facing surface, wherein the upwardly facing surface (i.e. the catch) of the lid is arranged to be brought into engagement with the downwardly facing surface of the base component so as to releasably fix the lid to the base component. This design of the lid provides an integrated catch with minimal complexity and thus increases the ease of cleaning. The production of such a catch is also straightforward as only a single component (the metal sheet) is needed for its construction.

In a very simple design, the entire side surface of the lid can form the clasp. However, in a preferred embodiment the one or more clasp is formed between two slits in the sheet that extend from the lower edge of the side surface towards or to the horizontal surface. This increases the flexibility of the clasp, while maintaining the simple, integral production of the clasp.

Preferably the downwardly facing surface of the base component is formed by a cut out in the side surface. However, this could alternatively by formed by the bottom edge of the side surface or underside of the horizontal surface.

According to a particularly preferred embodiment, the cassette is a metal-only cassette, which means that all components of the cassette are made of metal or a metal alloy. Most preferably, the metal is stainless steel, aluminium or titanium as these metals have particularly suitable characteristics, such as durability, rigidity, stain-resistance, bacteria-resistance, chemical inertness and oxidation resistance.

Particularly preferred embodiments of the present invention shall now described, by way of example only, with reference to the accompanying drawings in which purely schematically:

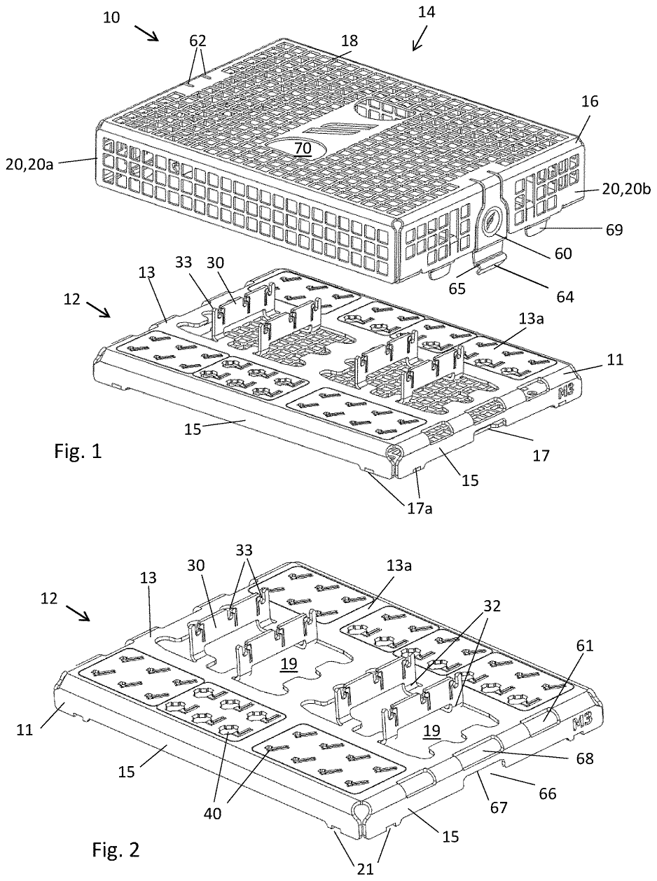

FIG. 1 shows in a perspective exploded view a first embodiment of the inventive cassette, in which a base component is arranged at a distance to a lid;

FIG. 2 shows a perspective top view of the inventive metal sheet of the base component of FIG. 1;

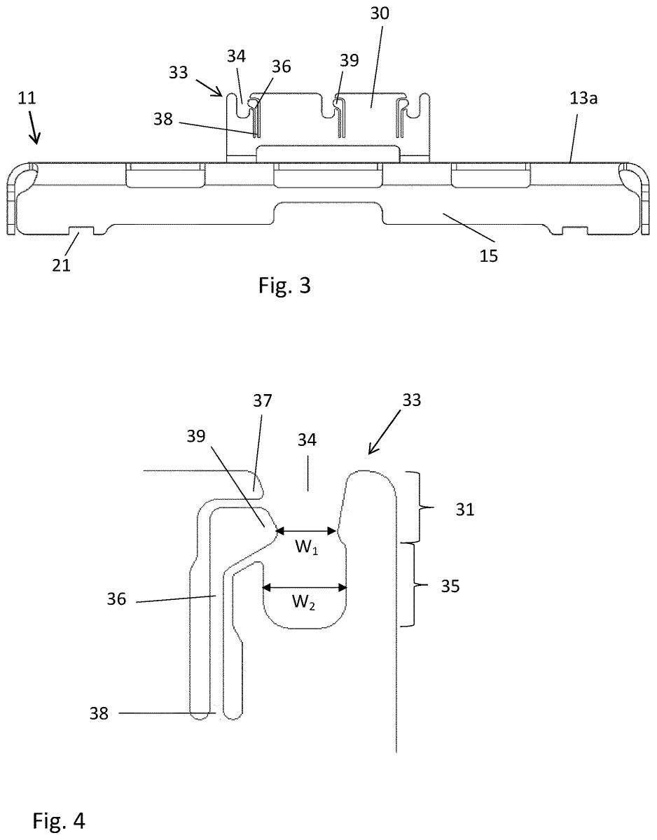

FIG. 3 shows a side view of the metal sheet of FIG. 2;

FIG. 4 shows a close up of the holding means of FIG. 3;

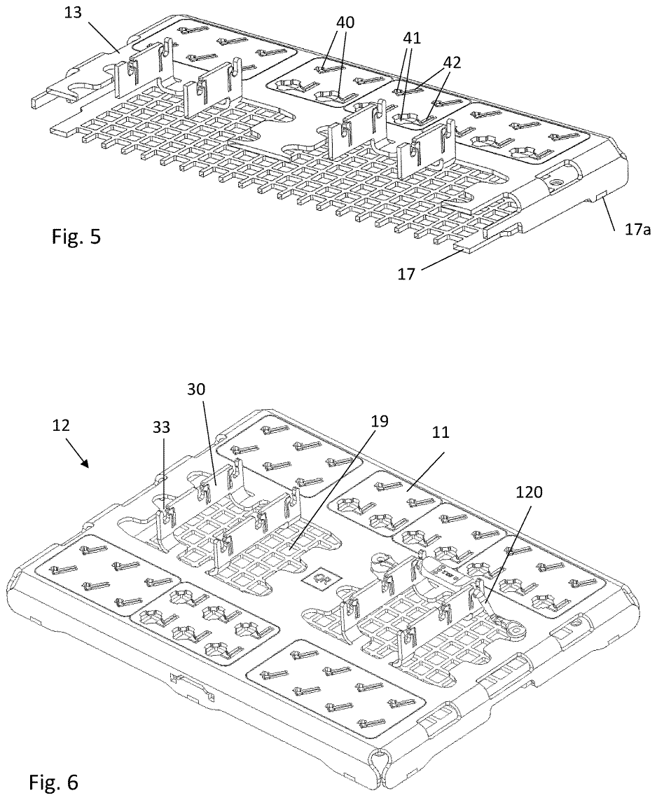

FIG. 5 shows in a perspective view a section through the base component of FIG. 1;

FIG. 6 shows the base component of FIG. 1 in combination with a surgical instrument:

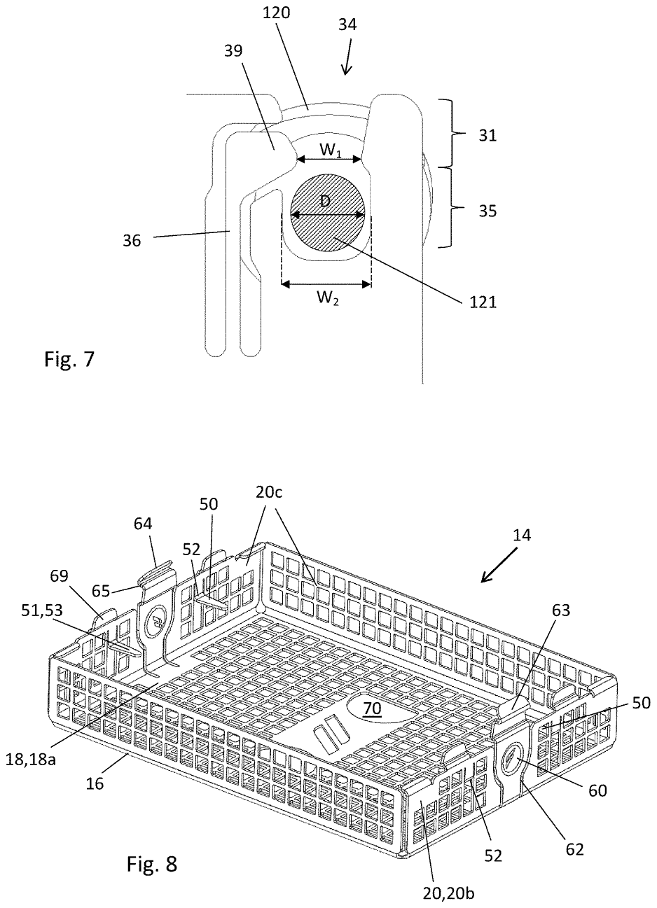

FIG. 7 shows the holding means of FIG. 4 together with a cross-section of the surgical instrument retained by the holding means;

FIG. 8 shows in a perspective view the lid of FIG. 1 turned upside down;

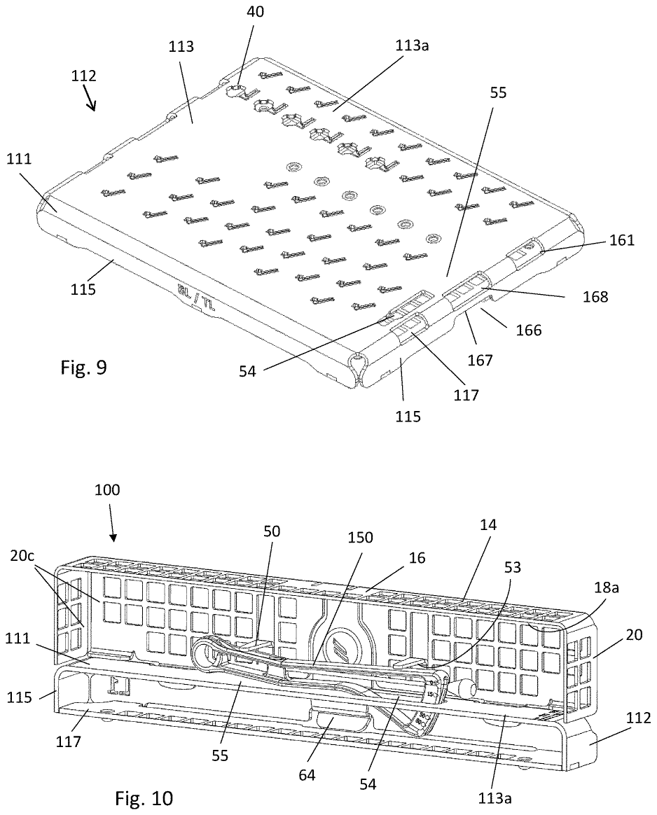

FIG. 9 shows an alternative base component which can be used with the lid of FIG. 1, and

FIG. 10 shows in a perspective view a section through the base component of FIG. 9 and lid of FIG. 1 in the assembled closed state, the cassette including an instrument held within the cassette.

FIG. 1 shows an embodiment of a cassette 10 according to the present invention, wherein the cassette 10 comprises a base component 12 and a lid 14. The lid 14 and the base component 12 are designed such that the lid can be releasably fastened to the latter, forming an enclosed space in which surgical instruments can be held for cleaning, sterilization, transport, surgical use and storage.

The lid 14 is formed entirely by a single metal sheet 16, the sheet 16 having a constant thickness which is considerably smaller than its length and width. This sheet 16 has a horizontal section 18 and four side sections 20, the side sections 20 being formed by end portions of the metal sheet 16 which extend downwards at approximately 90° to the horizontal section 18. In the shown embodiment the horizontal section 18 is rectangular in shape, thus resulting in mutually opposing long side sections 20a and short side sections 20b. However, the horizontal section 18 can have numerous alternative shapes, such as square, triangular etc. Horizontal section 18 has a lower horizontal surface 18a and side sections 20 have inner side surfaces 20c, as best seen in FIG. 8.

The base component 12 includes a (first) metal sheet 11 having a planar horizontal section 13 and four side sections 15, the side sections 15 being formed by end portions of the metal sheet 11 extending downwards at approximately 90° to the horizontal section 13. Horizontal section 13 of the base component 12 matches the size and shape of the horizontal section 18 of the lid 14 and comprises an upper horizontal surface 13a. Base component 12 further includes a second metal sheet 17 that is positioned essentially parallel to and at a distance below the horizontal section 13. The first and second metal sheets 11, 17 of the base component 12 may be welded or otherwise fixedly or detachably connected together, such that they can form a single unit.

All shown components of the cassette, including the lid 14 and the base component 12, are made of metal or a metal alloy. In the shown embodiments of the cassette there are thus no non-metal parts present.

When the lid 14 is fastened to the base component 12, an enclosed space is formed between the upper horizontal surface 13a of the base component 12, and the lower horizontal surface 18a and inner side surfaces 20c of the lid 14. Thus, these surfaces 13a, 18a and 20c are boundary forming surfaces of the metal sheets 16, 11 which together entirely delimit the enclosed space.

FIG. 2 shows in a perspective top view the metal sheet 11 of base component 12. This metal sheet 11 comprises a plurality of integral tongues 30, each tongue 30 forming a plurality of holding means 33 for retaining surgical instruments within the cassette 10. The tongues 30 are integrally connected to the boundary forming surface, namely upper horizontal surface 13a, of the metal sheet 11 at attachment locations 32 and extend from these attachment locations 32 into the enclosed space.

Each tongue 30 consists of a partially detached cut out in the sheet 11 which has been bent out of alignment with the boundary forming surface 13a. In the shown embodiment, the metal sheet 11 comprises four tongues 30 that are each connected to the boundary forming surface 13a at two attachment locations 32 located at discrete locations. Tongues 30 each extend in a single plane, which is approximately perpendicular to the boundary forming surface 13a. Further, the tongues 30 each comprise a plurality of holding means 33—best seen in FIGS. 3 and 4. The holding means 33 each comprise a passage 34 through the thickness of the tongue 30 with an integral coplanar spring element 36 adjacent to the passage 34. Each passage 34 and spring element 36 forms a self-sufficient holding means 33. The spring elements 36 consist of a section of the tongue 30 which is partially but not fully detached from the tongue 30 and which is integrally connected to the tongue 30 at a proximal attachment region 38. The spring elements 36 are coplanar to the tongue 30 at least when they are in their resting state. The spring elements 36 can be deflected within the plane of the tongue 30 in order to resiliently retain an instrument within the adjacent passage 34. Further details of the passage 34 and spring element 36 can be seen in FIG. 4.

As shown in FIG. 4, the passage 34 includes a distal insertion section 31 that is open towards a distal free end of the tongue 30 and a proximal holding section 35. The distal insertion section 31 thus forms an opening through which a surgical instrument can be inserted. In this embodiment the insertion section 31 has a width that decreases in the proximal direction in order to centre and guide the instrument during insertion. In the present embodiment the spring elements 36 are arranged to provide a passive hold on the instrument. The minimum width W₁ of the insertion section 31 is created by a detent 39 of the adjacent spring element 36 which detent protrudes into the passage 34 when the spring element 36 is in a resting state. The minimum width W₁ of the insertion section 31 is less than a maximum width W₂ of the holding section 35. The spring element 36 is elastically deflectable from the resting state shown in FIG. 4 into an active state to increase the minimum width W₁ of the insertion section 31. The surgical instrument (not shown) is pressed into the insertion section 31 of passage 34 causing the spring element 36 to deflect and the detent 39 to move outwards from the passage 34 in order to increase the width minimum W₁ of the insertion section 31. When the surgical instrument has passed the detent 39 the spring element 36 returns to its resting state and the user is provided with a physical sensation that informs them that the surgical instrument has now reached holding section 35.

In this embodiment the spring element 36 does not extend to the distal end of passage 34. Instead, the insertion section 31 comprises a section 37 distal of and partially covering the spring element 36 which protects the spring element 36 from damage.

Each tongue 30 comprises three self-sufficient holding means 33 of the type described above. The tongues 30 are arranged essentially parallel to one another and are spaced along the length of the horizontal section 13 of metal sheet 11. Each passage 34 of one tongue 30 is aligned with a passage 34 of another tongue 30 such that one surgical instrument can be held in a plurality of passages 34 each located in separate tongues 30. This allows an instrument to be retained at multiple points along its length and thus prevents tilting within the surgical cassette 10, see FIG. 6.

In addition to the above-described holding means 33, the metal sheet 11 additionally comprises a plurality of secondary holding means 40 for retaining surgical instruments (see FIG. 5). These holding means 40 include differently sized apertures 41 having a closed cross-section and having an adjacent spring element 42. These holding means are described in detail in WO 2016/142331 A1, the disclosure of which with respect to the design of these holding means is hereby included by reference.

Although not shown in the figures, the upper horizontal surface 13a of the base component 12 may be marked to assist the surgeon with identification of the instruments and the order or sequence of use of the surgical instruments accommodated within holding means. For instance, pictograms may be provided to ensure the correct pairing of surgical instrument and holding means. Such pictograms can be provided on the upper horizontal surface 13a of the base component by e.g. silk printing.

As can be seen from FIG. 5, similar to the lid 14, the second metal sheet 17 of base component 12 is perforated such that a cleaning fluid and sterilising gases can flow through the cassette 10, even in a closed state. These perforations allow the cassette with the at least one surgical instrument stored therein to be cleaned, e.g. in an ACD machine, and then sterilized, e.g. in an autoclave. In the first metal sheet 11 the holes 19 left behind in the boundary forming surface 13a after formation of the tongues 30 provide passages for sterilizing steam in the vicinity of the tools held by the tongues 30. In addition, as the instruments are held within the holding means 33 of the tongues 30 in a passive manner, steam can also pass around the instrument shafts while they are held within the passages 34.

The second metal sheet 17 is shaped as a planar perforated sheet that is connected to the bottom edges of the downwardly extending side sections 15 of the first metal sheet 11 of the base component 12. For connecting the two metal sheets 11, 17, the second metal sheet 17 comprises protrusions 17a (FIGS. 1 and 5) which fit into respective cut-outs 21 (FIGS. 2 and 3) in the bottom edges of the side sections 15 on the first metal sheet 11.

FIG. 6 shows the base component 12 with a surgical instrument 120 retained by holding means 33 in two separate tongues 30. FIG. 6 thus illustrates how a single instrument 120 can be retained by multiple self-sufficient holding means 33 at different locations along its length. As can also be clearly seen, holes 19 in the metal sheet 11 allow for a good flow of sterilization steam around instrument 120.

FIG. 7 shows a close up of holding means 33 while retaining instrument 120. Here it can be seen that the instrument shaft 121 is located in the proximal holding section 35 of passage 34, having passed through insertion section 31. The diameter D of shaft 121 is greater than the minimum width W₁ of the insertion section 31, created when spring element 36 is in its resting state. Thus, the instrument 120 is retained in the passage 34 but in a passive manner such that fluid can flow around shaft 121. Further, the diameter D of the shaft 121 is less than the maximum width W₂ of the holding section 35, which also assists with good fluid (both liquid and gaseous) circulation around the shaft 121.

FIG. 8 shows in a perspective view the lid 14 of FIG. 1 turned upside down. From this view, it is clearly visible that the lid 14 comprises four integral tongues 50, these tongues 50 forming holding means 51 for retaining one or more surgical instrument within the cassette (see FIG. 10). The tongues 50 each consist of a section of the metal sheet 16 which is partially but not fully detached from the sheet 16 and which is integrally connected to the boundary forming surface 20c at an attachment location 52. The tongues 50 are each formed by a cut out in the boundary forming surface 20c, this cut out being bent out of alignment with the boundary forming surface 20c at the attachment location 52 such that the tongue 50 extends from the attachment location 52 into the enclosed space of the cassette 10. Each tongue 50 extends at an angle of approximately 90° relative to the plane of the metal sheet 16 at the attachment location 52, the tongues 50 each extending in a single plane. In contrast to the tongues 30 of the base component 12, the tongues 50 of the lid 14 form cooperative holding means. Each holding means 51 comprises a holding surface 53 for retaining an instrument, this holding surface 53 opposing a cooperative surface 55 of a base component 112, as will described below in relation to FIGS. 9 and 10.

FIG. 9 shows an alternative base component 112, which can be used with the lid 14 (see FIG. 1) to create cassette 100. The alternative base component 112 includes a (first) metal sheet 111 having a planar horizontal section 113 and four side sections 115, the side sections 115 being formed by end portions of the metal sheet 111 extending downwards at approximately 90° to the horizontal section 113. The horizontal section 113 of the base component 112 matches the size and shape of the horizontal section 18 of the lid 14 and comprises an upper horizontal surface 113a. The base component 112 further includes a second metal sheet 117 that is positioned essentially parallel to and at a distance below the horizontal section 113. The first and second metal sheets 111, 117 of the alternative base component 112 may be welded or otherwise fixedly or detachably connected together, such that they can form a single unit.

In contrast to the base component 12 (shown in FIGS. 1-6), the (alternative) base component 112 does not comprise tongues 30, although it does contain the same secondary holding means 40 as described in relation to base component 12. Instead, the upper horizontal surface 113a forms a cooperating surface 55 which opposes the holding surfaces 53 formed by tongues 50 of lid 14 (FIGS. 1 and 8). The cooperating surface 55 comprises an aperture 54 for housing a part of a surgical instrument 150.

FIG. 10 shows a section through the cassette 100 comprising the (alternative) base component 112 of FIG. 9 and the lid 14 of FIGS. 1 and 8 in the closed or “assembled” state. The upper horizontal surface 113a of the base component 112 and the lower horizontal surface 18a and the inner side surfaces 20c of the lid 14 form boundary forming surfaces of the metal sheets 111, 16 which together entirely delimit an enclosed space in which the surgical instruments are stored.

FIG. 10 shows how two tongues 50 of the lid 14 hold a surgical instrument 150 in place within the cassette 100. In the shown embodiment, part of the handle of the surgical instrument 150 is fitted into the aperture 54 in the cooperating surface 55 of the (alternative) base component 112, while other regions of the surgical instrument 150 rest on the cooperating surface 55. In the assembled state of the cassette 100, the holding surfaces 53 of the two tongues 50 of the lid 14 lie in close proximity to the top surface of the surgical instrument 150 and thus prevent the instrument 150 from shifting out of position within the cassette 100.

For securing the lid 14 on the base component 12, 112 the former comprises integral clasps 60, which are configured to couple with the base component 12, 112.

More specifically, as best seen in FIGS. 1 and 8, the lid 14 comprises two integral clasps 60. Each clasp 60 is formed between two slits 62 in the short side sections 20b of the sheet 16, the slits 62 extending from the lower edge of the side sections 20b to the horizontal section 18. The clasps 60 are pivotable relative to the horizontal section 18 of the lid 14, each clasp 60 comprising a catch 64. The catch 64 is formed by a portion of the metal sheet 16 which is bent out of the plane of the clasp 60 so as to form an upwardly facing surface 65. In the present embodiment this is achieved by creating an S shaped latch 63 at the distal end of the clasp 60. The side sections 15, 115 of the metal sheet 11, 111 of the base component 12, 112 comprise cut outs 66, 166 (FIG. 2, 9) which form a downwardly facing surface 67, 167. The upwardly facing surface 65 of the catch 64 is arranged to be brought into engagement with the downwardly facing surface 67, 167 of the base component 12, 112 so as to releasably fix the lid 14 onto the base component 12, 112. In this preferred embodiment the clasp 60 passes through a cut out 68, 168 in the metal sheet 11, 111 of the base component 12, 112 before reaching the downwardly facing surface 67, 167. In addition, each short side section 20b (FIG. 1) comprises two small tab-like vertical protrusions 69. These protrusions 69 are intended to fittingly extend into corresponding cut-outs 61, 161 provided in the upper edge of the base component 12, 112. In this way, the side sections 20b of the lid 14 and the side sections 15, 115 of the base component 12, 112 are secured and aligned with respect to each other in a simple manner.

The lid 14 further comprises two D-shaped cut-outs 70 in its planar horizontal section 18. These cut-outs 70 enable a user's fingers to be inserted therein to lift the cassette 10, 100, e.g. out of a sterilisation container or transportation package. Thanks to the D-shaped cut-outs 70, no handle is required, which avoids areas of overlapping material and therefore facilitates cleaning and sterilization of the cassette 10, 100.

The above described embodiments are for illustrative purposes only and the skilled person will realize that alternative arrangements are possible which fall within the scope of the claims. For example, in other embodiments tongues on the lid may form self-sufficient holding means and tongues on the base component may form cooperative holding means. The base component may comprise side sections that form boundary forming surfaces of the enclosed space. The tongues may extend from the attachment locations at angles other than 90° and may comprise integral spring elements which do not extend in the plane of the tongue.

1. A metal cassette for housing at least one surgical instrument, the cassette comprising
a base component and a lid fastenable to the base component to form an enclosed space in which the at least one surgical instrument can be housed,
at least one of the base component and lid comprising a metal sheet, the enclosed space being partly delimited by a boundary forming surface of the metal sheet,
the metal sheet comprising at least one integral tongue forming at least one holding means for retaining the at least one surgical instrument within the cassette, the tongue consisting of a section of the metal sheet which is partially but not fully detached from the metal sheet and which is integrally connected to the boundary forming surface at at least one attachment location, wherein, in a resting state, the tongue extends from the at least one attachment location into the enclosed space.

2. The metal cassette as claimed in claim 1, wherein the at least one tongue extends from the at least one attachment location at an angle of between 30°-150°, relative to the plane of the metal sheet at the at least one attachment location of the tongue.

3. The metal cassette as claimed in claim 1, wherein the at least one tongue is formed by a partially detached cut out in the boundary forming surface of the metal sheet, the cut out being bent out of alignment with the boundary forming surface at the at least one attachment location.

4. The metal cassette as claimed in claim 3, wherein the at least one tongue is formed by a cut out in a section of the boundary forming surface which extends in a single plane.

5. The metal cassette as claimed in claim 1, wherein the at least one tongue extends in a single plane.

6. The metal cassette as claimed in claim 1, wherein the at least one tongue forms at least one holding means comprising a holding surface for retaining an instrument, the holding surface opposing a cooperating surface of the cassette not located on the tongue such that in use the instrument is retained between the holding surface and the cooperating surface.

7. The metal cassette as claimed in claim 6, wherein the holding surface of the tongue is planar and the cooperating surface comprises a cavity, depression or aperture for housing the instrument.

8. The metal cassette as claimed in claim 1, wherein the at least one tongue forms at least one holding means comprising at least one integrally formed spring element, moveable relative to the remainder of the tongue, for resiliently retaining an instrument between the at least one spring element and another area of the tongue.

9. The metal cassette as claimed in claim 8, wherein the at least one spring element consists of a section of the tongue which is partially but not fully detached from the tongue and which is integrally connected to the tongue at a proximal attachment region, the at least one spring element extending in the plane of the tongue, such that the spring element is coplanar to the tongue when it is in the resting state.

10. The metal cassette as claimed in claim 9, wherein the at least one holding means further comprises a passage through the thickness of the tongue, the at least one integral coplanar spring element being adjacent to the passage.

11. The metal cassette as claimed in claim 10, wherein the passage includes a distal insertion section that is open towards a distal free end of the tongue for allowing insertion of a surgical instrument into the passage and a proximal holding section intended for releasably housing the surgical instrument.

12. The metal cassette as claimed in claim 1, wherein the at least one tongue is arranged to provide a passive hold on the instrument.

13. The metal cassette as claimed in claim 1, wherein the metal sheet comprises a plurality of integral tongues, each forming at least one holding means for retaining at least one surgical instrument.

14. The metal cassette as claimed in claim 1, wherein the metal sheet comprises a planar horizontal boundary forming surface, the planar horizontal boundary forming surface delimiting the full horizontal extent of the enclosed space.

15. The metal cassette as claimed in claim 1, wherein both the base component and lid comprise a metal sheet in accordance with claim 1.

16. The metal cassette as claimed in claim 1, wherein the lid comprises a metal sheet according to claim 1, whereby the sheet comprises a planar horizontal surface and two or more side surfaces, the side surfaces being formed by end portions of the metal sheet bent at 90° to the plane of the horizontal surface, the horizontal and side surfaces forming a boundary forming surface of the enclosed space, the lid further comprising one or more integral clasp, the clasp being formed by a side surface of the lid, wherein the clasp is pivotable relative to the horizontal surface, the clasp comprising a catch, the catch being formed by a portion of the metal sheet which is bent out of the plane of the clasp so as to form an upwardly facing surface,
the base component comprising one or more side walls, the one or more side wall comprising a downwardly facing surface, wherein the upwardly facing surface of the lid is arranged to be brought into engagement with the downwardly facing surface of the base component so as to releasably fix the lid to the base component.
